(19) United States
(12) Patent Application Publication
(10) Pub. No.: US 2015/0342763 A1
(43) Pub. Date: Dec. 3, 2015

(54) INTRAVASCULAR STENT WITH REGIO-SELECTIVE MATERIALS AND STRUCTURES

(71) Applicant: National Taiwan University, Taipei City (TW)

(72) Inventor: Hao-Ming HSIAO, Taipei City (TW)

(21) Appl. No.: 14/678,194

(22) Filed: Apr. 3, 2015

(30) Foreign Application Priority Data
May 27, 2014 (TW) 103118364

(51) Int. Cl. A61F 2/90; A61L 31/06; A61L 31/02

(52) U.S. Cl. CPC A61F 2/90; A61L 31/022; A61L 31/06; B33Y 80/00

(57) ABSTRACT

The present invention relates to a stent, comprising: a plurality of radially-expandable rings arranged along a longitudinal axis, wherein each of the radially-expandable rings may include a plurality of bar arms and a plurality of crowns, the adjacent crowns being connected by the bar arms therebetween, and a plurality of connectors being disposed in between and connecting the radially-expandable rings, wherein the bar arms may include a first material, and the crowns may include a second material which may be different from the first material. Novel techniques for manufacturing a stent, such as 3D additive printing methods, could be used for realizing the disclosed stents.

### CROSS REFERENCE TO RELATED APPLICATION

This application claims the benefits of Taiwan Patent Application Serial Number 103118364, filed on May 27, 2014, the subject matter of which is incorporated herein by reference.

### BACKGROUND OF THE INVENTION

1. Field of the Invention

The present invention relates to a stent, and especially to a stent with regio-selective materials and structures.

2. Description of Related Art

Vascular stenting has become one of the standard treatments for cardiovascular disease, and with the ever-changing medical technologies, a variety of stent-related inventions, such as drug-eluting stents, bioresorbable stents, and the like, are widely used for treating various cardiovascular diseases, such as coronary and peripheral artery diseases.

For the clinical application of the vascular stent, important factors in stent design are deliverability, radial strength, flexibility, conformability, fracture toughness, and fatigue resistance. Therefore, conventional stents made of metals or alloys have been used in clinical practice due to their good clinical outcome. However, due to their long-term presence in the body, the use of such metallic stents has raised concerns about thrombosis. To alleviate such concerns, stents made of bioresorbable materials have been developed to reduce the problem of thrombosis.

The stresses of a stent are concentrated almost entirely on the crowns or the connectors, while the bar arms bear almost no stresses. Due to the limitations of stent manufacturing techniques (such as laser cutting), conventional solutions typically re-arrange the stress distribution of a vascular stent by variations in the design patterns or geometric parameters. However, the changes to the design patterns or geometric parameters of the stent still have their limitations when conventional stent manufacturing techniques are applied. Furthermore, in a bioresorbable stent, a great amount of stress tends to concentrate in the crowns and can cause in cracks and fractures due to the inherent properties of polymer materials. Compared to those of metallic stents, the fracture toughness of the polymeric stent is insufficient and must be improved.

3D additive printing is a rapid prototyping technology wherein a product is constructed layer-by-layer of an adhesive material such as a powdered metal or plastic. This method allows quick printing, significantly reduces the consumption of the raw materials, and is customizable. 3D additive printing is considered to have the potential to reshape future medical industries. One new trend in medical development is the production of a 3D printed stent. In addition to allowing customization of stents for patients, 3D additive printing can also break through the limitations of traditional stent design, allowing ideas that were unfeasible in the past to be realized. The intravascular stent with regio-selective materials and structures of the present invention is one example thereof.

Therefore, according to the clinical needs, it is desirable to provide an intravascular stent with regio-selective materials and structures that has the properties of high radial strength, good deliverability, high flexibility, and high fracture toughness.

### SUMMARY OF THE INVENTION

An object of the present invention is to provide a stent whose properties at specific locations are improved by adjusting the local composition of the stent materials, thereby providing a stent with high radial strength, good deliverability, high fracture toughness and fatigue resistance.

To achieve the above object, the present invention provides a stent comprising: a plurality of radially-expandable rings arranged along a longitudinal axis, wherein each of the radially-expandable rings includes a plurality of bar arms and a plurality of crowns, the adjacent crowns being connected by the bar arms therebetween, and a plurality of connectors being disposed in between and connecting the radially-expandable rings, wherein the bar arms include a first material, and the crowns include a second material which is different from the first material.

As described above, the material composition ratio of the stent of the present invention is altered at the positions where stress is concentrated (e.g., the crowns and the connectors) to guide the stress to the bar arms, which bear no force originally, thereby improving the stress concentration and lifespan of the stent. In addition, a third phase is added or generated in the positions where stress is concentrated to improve the fracture toughness so as to produce a stent having both high radial strength and fracture toughness. The above technical features can be applied to various types of stents, and the present invention is not particularly limited thereto. Preferably, in the stent of the present invention, the radially-expandable ring may include the bar arms and the crowns, and the adjacent crowns may be connected by the bar arms. Specifically, in an embodiment of the present invention, each of the radially-expandable rings may be composed of the plurality of bar arms and the crowns, and the adjacent crowns may be connected by the bar arms. In other words, the bar arms and the crowns may be alternately arranged and connected to each other to form each of the radially-expandable rings. More preferably, in an exemplary embodiment of the present invention, the bar arms and the crowns may be alternately arranged and connected to each other in a continuous wavy shape to form each of the radially-expandable rings. However, as long as the above-described object of the present invention can be achieved, one having ordinary skill in the art can use any conventional vascular stent design, and the present invention is not particularly limited thereto.

In the stent of the present invention, since the bar arms and the crowns comprise different materials respectively, then considering the binding degree between the bar arms and the crowns, preferably, in an embodiment of the present invention, a content of a first material gradually decreases from 100 wt % to 0 wt % from the bar arms to the adjacent crowns. In another embodiment of the present invention, a content of a second material gradually decreases from 100 wt % to 0 wt % from the crowns to the bar arms. Furthermore, and more preferably, in a further embodiment of the present invention, a content of the first material gradually decreases from 100 wt % to 0 wt % from the bar arms to the adjacent crowns, while a content of the second material gradually decreases from 100 wt % to 0 wt % from the crowns to the bar arms. Most preferably, the plurality of bar arms and the crowns may be composed of a first material and a second material respectively, wherein a content of the first material gradually decreases from 100 wt % to 0 wt % from the bar arms to the adjacent crowns in a graded distribution and a content of the second material gradually decreases from 100 wt % to 0 wt % from the crown to the bar arm in a graded distribution, but the present invention is not limited thereto. In addition, the bar arms and the adjacent crowns each may independently contain other identical or compatible materials to avoid a large discrepancy in material properties between the bar arms and the adjacent crowns, which may result in insufficient binding. For example, the bar arms and the adjacent crowns each may comprise a third material compatible with both the first material and the second material. Alternatively, the bar arms may further comprise a third material, while the adjacent crowns may further comprise a fourth material which can be compatible with the third material, but the present invention is not limited thereto.

In the stent of the present invention, as long as the object of improving the radial strength and fracture toughness of the local position of the stent can be achieved by locally adjusting the composition of the materials, the materials constituting each local part of the stent in the present invention are not particularly limited. Specifically, in an embodiment of the present invention, the first material may comprise a component A, and based on the total weight of the bar arms, a content of the component A is 50-100 wt %; the second material may comprise a component B, and based on the total weight of the crowns, a content of the component B is 50-100 wt %. Preferably, in another embodiment of the present invention, the first material may further include a component B, and the weight ratio of the component B to the component A (B/A) may be greater than 0 and less than or equal to 1. In still another embodiment of the present invention, the second material may further comprise a component A, and a weight ratio of the component A to the component B (A/B) may be greater than 0 and less than or equal to 1. Alternatively, in a further embodiment of the present invention, the first material may include the component A and the component B, and based on the total weight of the bar arms, the content of the component A may be 50-100 wt %, and the weight ratio of the component B to the component A (B/A) may be greater than 0 and less than or equal to 1; the second material may include the component A and the component B, and based on the total weight of the crowns, the content of component B may be 50-100 wt %, and the weight ratio of the component A to the component B (A/B) may be greater than 0 and less than or equal to 1. In other words, the bar arms and the crowns may each independently include the component A and the component B, and the bar arms may be mainly composed of the component A, while the crowns may be mainly composed of the component B. Furthermore, in an exemplary embodiment of the present invention, the weight ratio of the component B to the component A (B/A) from the bar arm to the crown may have a graded distribution. In another specific embodiment of this present invention, the weight ratio of the component A to the component B (A/B) from the crown to the bar arm has a graded distribution. In other words, in the bar arms, the crowns, or both, the proportion of the component A and the component B may be incremental or decremental in a direction therebetween, but the present invention is not limited thereto. More preferably, in an exemplary embodiment of the present invention, the weight ratio of the component B to the component A (B/A) from the bar arm to the crown may have a continuous graded distribution, while the weight ratio of the component A to the component B (A/B) from the crown to the bar arm has a continuous graded distribution. Accordingly, in this exemplary embodiment of the present invention, the bar arms may be mainly composed of the component A, while the crowns may be mainly composed of the component B, and the weight ratio of the component B to the component A (B/A) from the center of the bar arm to the center of the crown may have a continuously incremental graded distribution, so as to achieve the object of improving the radial strength and fracture toughness of the local position of the stent by locally adjusting the composition of the materials.

In the present invention, the term “graded distribution” refers to the case where a weight ratio of the material composition exhibits an incremental or decremental distribution in a specific direction, wherein the gradation may include the case of a gradient or a continuous variation. Furthermore, when the material composition has a gradient distribution, the differences between the grades may be the same or different, and when the material composition is distributed in a continuous variation, the variance may be changed in a form of various function curves (such as an arithmetic progression, a geometric progression, a parabola, or a trigonometric function), and the present invention is not particularly limited thereto.

In the stent of the present invention, as long as the radial strength and fracture toughness of the local position may be improved by locally adjusting the composition of the materials, various types of materials can be used for the stent, and the present invention is not particularly limited thereto. For example, in an embodiment of the present invention, the component A and the component B are different, and each independently is a metal, an alloy, a polymer, or a combination thereof. Preferably, in an exemplary embodiment of the present invention, the component A and the component B may be different and each independently is nickel, titanium, cobalt, tantalum, chromium, platinum, magnesium, iron, alloys thereof, stainless steel, or any combination thereof. More preferably, in another specific embodiment of the present invention, the component A and the component B may be different and each independently is poly(L-lactide) acid (PLLA), polyglycolic acid (PGA), polycaprolactone (PCL), poly(DL-lactide) acid (PDLLA), polydioxanone (PDS), or a combination thereof.

In the stent of the present invention, in order to improve the fracture toughness of the local position of the stent (e.g., a position where stress is concentrated), in addition to the component A and the component B, the second material may further include a component C. Specifically, in an embodiment of the present invention, the component C may be a glass fiber, a carbon fiber, a microparticle, a nanoparticle, a crosslinking agent, a curing agent, or a combination thereof, which may be produced by artificial addition or a cross-linking reaction, but the invention is not limited thereto.

In the stent of the present invention, as long as the object of improving the deliverability of the local position of the stent can be achieved, various types of materials may be used to prepare the connectors of the stent of the present invention. Specifically, in an embodiment of the present invention, the connectors of the stent of the present invention may comprise a first material, a second material, a combination thereof, or other materials (such as the aforementioned third and fourth materials), but the present invention is not particularly limited thereto. Preferably, the connectors may comprise a first material, a second material, or a combination thereof. Furthermore, in the connectors, a proportion of a first material, a second material, or a combination thereof may also have a graded distribution. More preferably, the connectors of the stent of the present invention may comprise a first material and a second material, and a proportion of the first material and the second material has a graded distribution in the connectors, but the invention is not limited thereto.

In addition, as long as the object of improving the deliverability of the local position of the stent can be achieved, the shapes of the crowns, the bar arms, the connectors and so on of the stent in the present invention are not particularly limited. For example, in an embodiment of the present invention, the crowns may have a U-shape, a Y-shape, a W-shape, or a combination thereof, but the present invention is not limited thereto. In another embodiment of the present invention, the connectors may have a cylindrical shape, a polygonal column shape, a spring-like shape, or a combination thereof, and preferably a spring-like shape, wherein the cross-sections of the connectors may be solid or hollow, but the present invention is not particularly limited thereto.

Furthermore, in the stent of the present invention, as long as a stent having different material compositions at local positions can be prepared, any preparation methods can be used, and the present invention is not particularly limited thereto. Preferably, in the embodiment of the present invention, the stent of the present invention may be prepared by a 3D additive printing method.

### BRIEF DESCRIPTION OF THE DRAWINGS

FIG. 1A shows a three-dimensional schematic drawing of the vascular stent 1 according to Example 1 of the present invention.

FIG. 1B is an enlarged view of the part A of the vascular stent 1 according to Example 1 of the present invention.

FIG. 2A shows a three-dimensional schematic drawing of the vascular stent 2 according to Example 2 of the present invention.

FIG. 2B is an enlarged view of the part B of the vascular stent 2 according to Example 2 of the present invention.

FIG. 3 is an enlarged partial view of the vascular stent 3 according to Example 3 of the present invention.

FIG. 4 is an enlarged partial view of the vascular stent 4 according to Example 4 of the present invention.

### DETAILED DESCRIPTION OF THE PREFERRED EMBODIMENT

In the following description, numerous specific details are set forth to provide a thorough understanding of embodiments of the present disclosure. However, one having ordinary skill in the art will recognize that embodiments of the disclosure can be practiced without these specific details. In some instances, well-known structures and processes are not described in detail to avoid unnecessarily obscuring embodiments of the present disclosure.

### Example 1

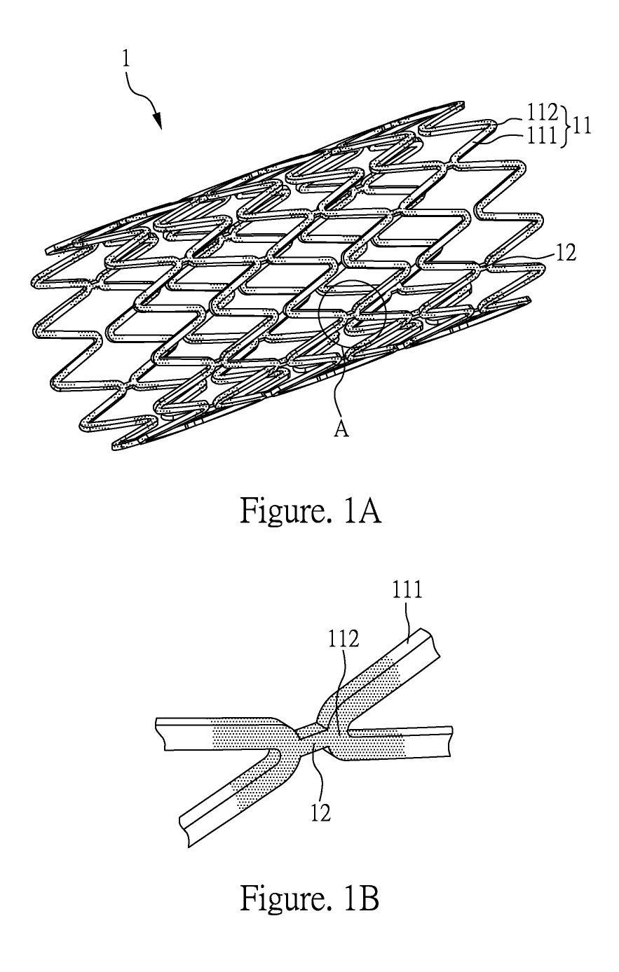

Please refer to FIGS. 1A and 1B, which illustrate a three-dimensional schematic drawing of the vascular stent 1 and an enlarged view of its part A according to Example 1 of the present invention. As shown in FIG. 1A, the stent 1 comprises: a plurality of radially-expandable rings 11 arranged along a longitudinal axis, wherein each of the radially-expandable rings 11 may include a plurality of bar arms 111 and a plurality of crowns 112, the adjacent crowns 112 being connected by the bar arms 111 therebetween, and a plurality of connectors 12 being disposed in between and connecting the radially-expandable rings 11. As shown in FIG. 1B, the bar arms 111 may include a first material, and the crowns 112 may include a second material which may be different from the first material. Furthermore, in this Example of the present invention, the connector 12 may be composed of a first material, a second material, or other materials, and the bar arms 111, the crowns 112, and the connector 12 can be an integrated structure.

In this Example of the present invention, the first material and the second material may each independently be composed of two or more bioresorbable polymer materials (e.g., poly(L-lactide) acid (PLLA), polyglycolic acid (PGA), polycaprolactone (PCL), poly(DL-lactide) acid (PDLLA), and polydioxanone (PDS)), and the second material may further include a component C which can improve the fracture toughness of the crowns 112 and the connectors 12 by an additive or a crosslinking reaction. However, as long as the fracture resistance of the crowns 112 and the connectors 12 can be improved, one having ordinary skill in the art may also use other materials as a component C (such as glass fibers, carbon fibers, and toughening particles), which may be formed into an organic-inorganic composite with the bioresorbable polymer materials of the component A and the component B to improve the fracture toughness of the crowns 112 and the connectors 12, but the present invention is not particularly limited thereto. Furthermore, in this Example of the present invention, the crosslinking agent used as the component C may be any conventional light cross-linking agent or thermal crosslinking agent capable of improving the fracture toughness of the local structure of the vascular stent by whole or partial light radiation or heating to initiate the cross-linking reaction.

In addition, in order to realize the above design of adjusting the material composition of the local structure, in this Example of the present invention, the 3D additive printing technique is employed to prepare the stent 1. Therefore, during the manufacturing, the material composition of the local structure of the stent is instantly adjusted to improve the radial strength and fracture toughness. In this Example of the present invention, one having ordinary skill in the art can appropriately adjust the processing parameters of the 3D additive printing technique in accordance with various characteristics such as the selected material composition, the required mechanical strength and so on, but the present invention is not particularly limited thereto. For clarity and brevity, the details of adjustments of the parameters are not described herein.

### Example 2

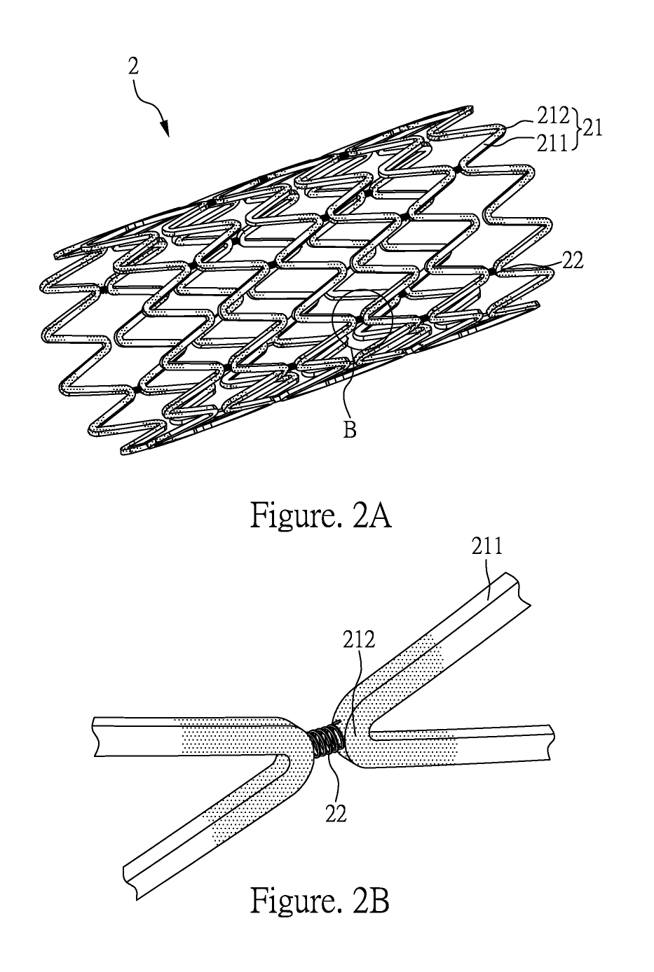

Please refer to FIGS. 2A and 2B, which illustrate a three-dimensional schematic drawing of the vascular stent 2 and an enlarged view of its part B according to Example 2 of the present invention. Example 2 and Example 1 are substantially the same, except that the connectors 22 of the stent 2 of Example 2 have a spring-like structure.

Therefore, as shown in FIG. 2A, the stent 2 comprises: a plurality of radially-expandable rings 21 arranged along a longitudinal axis, wherein each of the radially-expandable rings 21 may include a plurality of bar arms 211 and a plurality of crowns 212, the adjacent crowns 212 being connected by the bar arms 211 therebetween, and a plurality of connectors 22 being disposed in between and connecting the radially-expandable rings 21. As shown in FIG. 2B, the bar arms 211 may include a first material, and the crowns 212 may include a second material which may be different from the first material. Furthermore, in this embodiment of the present invention, the connector 22 may be composed of the first material, the second material, or other materials, and the bar arms 211 and the crowns 212 may have a columnar structure while the connector 12 may have a spring-like structure. The connector 22, the bar arms 211, and the crowns 212 can be formed integrally.

The material composition and preparation method of Example 2 are substantially the same as that of Example 1, and the detailed description is not repeated herein.

### Example 3

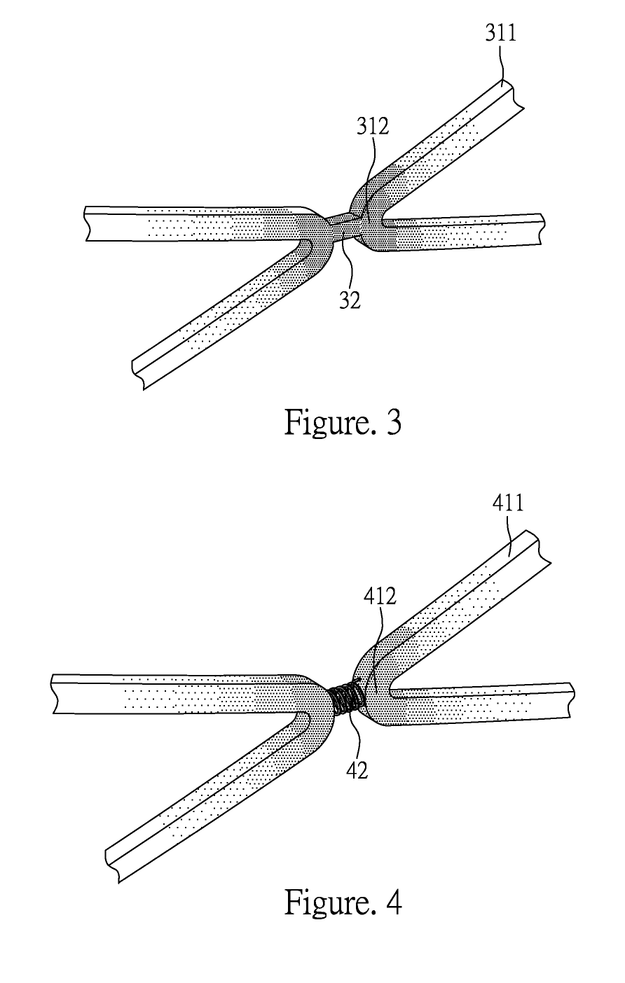

Example 3 is substantially the same as Example 1, except that the material composition of the crowns and the bar arm has a graded distribution. Specifically, refer to FIG. 3, which is an enlarged partial view of the vascular stent 3 according to Example 3. As shown in FIG. 3, the bar arm 311 is made of the first material, and the crown 312 and the connector 32 are made of the second material, wherein the first material and the second material comprise bioresorbable polymer materials (e.g., poly(L-lactide) acid (PLLA), polyglycolic acid (PGA), polycaprolactone (PCL), poly(DL-lactide) acid (PDLLA), and polydioxanone (PDS)), and the weight ratio of the component B to the component A (B/A) from the center of the bar arm 311 to the center of the crown 312 has an incremental graded distribution.

Accordingly, by the graded distribution of the material composition ratio, this Example of the present invention not only improves the radial strength of the local position of the stent but also solves the material compatibility problem due to the large discrepancy in the material composition, if existing.

The structure and preparation method of Example 3 are substantially the same as that of Example 1, and the detailed description is not repeated herein.

### Example 4

Example 4 is substantially the same as Example 2, except that the material composition of the crowns and the bar arm has a graded distribution. Specifically, please refer to FIG. 4, which is an enlarged partial view of the vascular stent 4 according to Example 4. As shown in FIG. 4, the bar arm 411 is made of a first material, and the crown 412 and the connector 42 are made of a second material, wherein the first material and the second material comprise bioresorbable polymer materials (e.g., poly(L-lactide) acid (PLLA), polyglycolic acid (PGA), polycaprolactone (PCL), poly(DL-lactide) acid (PDLLA), and polydioxanone (PDS)), and the weight ratio of the component B to the component A (B/A) from the center of the bar arm 411 to the center of the crown 412 has an incremental gradient distribution.

Accordingly, by the graded distribution of the ratio of material composition, this Example of the present invention not only improves the radial strength of the local position of the stent but also solves the material compatibility problem due to the large discrepancy in the material composition, if existing.

The structure and preparation method of Example 4 are substantially the same as that of Example 2, and the detailed description is not repeated herein.

It should be understood that these examples are merely illustrative of the present invention and that the scope of the invention should not be construed to be defined thereby; the scope of the present invention will be limited only by the appended claims.

What is claimed is:

1. A stent, comprising:
a plurality of radially-expandable rings arranged along a longitudinal axis, wherein each of the radially-expandable rings includes a plurality of bar arms and a plurality of crowns, adjacent crowns being connected by the bar arms therebetween; and
a plurality of connectors disposed in between and connecting the radially-expandable rings;
wherein the bar arms comprise a first material, and the crowns comprise a second material which is different from the first material.

2. The stent as claimed in claim 1, wherein a content of the first material gradually decreases from 100 wt % to 0 wt % from the bar arms to the adjacent crowns.

3. The stent as claimed in claim 1, wherein a content of the second material gradually decreases from 100 wt % to 0 wt % from the crowns to the adjacent bar arms.

4. The stent as claimed in claim 1, wherein the first material comprises 50-100 wt % of the component A, based on the total weight of the bar arms.

5. The stent as claimed in claim 1, wherein the first material further comprises a component B, and the weight ratio of the component B to the component A (B/A) is greater than 0 and less than or equal to 1.

6. The stent as claimed in claim 1, wherein the second material comprises 50-100 wt % of the component B, based on the total weight of the crowns.

7. The stent as claimed in claim 6, wherein the second material further comprises a component A, and a weight ratio of the component A to the component B (A/B) is greater than 0 and less than or equal to 1.

8. The stent as claimed in claim 5, wherein the weight ratio of the component B to the component A (B/A) from the bar arm to the crown has a graded distribution.

9. The stent as claimed in claim 7, wherein the weight ratio of the component A to the component B (A/B) from the crown to the bar arm has a graded distribution.

10. The stents of claim 5, wherein the component A and the component B are different and each independently is a metal, an alloy, a polymer, or a combination thereof.

11. The stents of claim 7, wherein the component A and the component B are different and each independently is a metal, an alloy, a polymer, or a combination thereof.

12. The stent as claimed in claim 10, wherein the component A and the component B are different, and each independently is nickel, titanium, cobalt, tantalum, chromium, platinum, magnesium, iron, alloys thereof, stainless steel, or any combination thereof.

13. The stent as claimed in claim 11, wherein the component A and the component B are different, and each independently is nickel, titanium, cobalt, tantalum, chromium, platinum, magnesium, iron, alloys thereof, stainless steel, or any combination thereof.

14. The stent as claimed in claim 10, wherein the polymer is poly(L-lactide) acid (PLLA), polyglycolic acid (PGA), polycaprolactone (PCL), poly(DL-lactide) acid (PDLLA), polydioxanone (PDS), or a combination thereof.

15. The stent as claimed in claim 11, wherein the polymer is poly(L-lactide) acid (PLLA), polyglycolic acid (PGA), polycaprolactone (PCL), poly(DL-lactide) acid (PDLLA), polydioxanone (PDS), or a combination thereof.

16. The stent as claimed in claim 7, wherein the second material further comprises a component C.

17. The stent as claimed in claim 16, wherein the component C is a glass fiber, a carbon fiber, a microparticle, a nanoparticle, a crosslinking agent, a curing agent, or a combination thereof.

18. The stent as claimed in claim 1, wherein each of the connectors independently comprises the first material, the second material, or a combination thereof.

19. The stent as claimed in claim 18, wherein a content of the first material, the second material, or a combination thereof in the connectors has a graded distribution.

20. The stent as claimed in claim 1, wherein the connector has a cylindrical shape, a polygonal column shape, a spring-like shape, or a combination thereof.

21. The stent as claimed in claim 1, wherein the stent is prepared by a 3D additive printing method.
